(19) United States
(12) Patent Application Publication
(10) Pub. No.: US 2026/0124061 A1
(43) Pub. Date: May 7, 2026

(54) FOLDABLE INFLATABLE LOAD BEARING STRUCTURE WITH METAL SUPPORT

(71) Applicant: Gil YOSEF, Givat Yearim (IL)

(72) Inventor: Gil YOSEF, Givat Yearim (IL)

(21) Appl. No.: 19/438,869

(22) Filed: Jan. 2, 2026

Related U.S. Application Data
(63) Continuation-in-part of application No. PCT/IL2024/050644, filed on Jul. 3, 2024.

(30) Foreign Application Priority Data
Jul. 5, 2023 (IL) 304279
Sep. 14, 2023 (IL) 305966

(51) Int. Cl. A61F 5/058; A61G 1/013; A61L 15/08; A61L 15/12; A61L 15/14

(52) U.S. Cl. CPC A61F 5/05816; A61G 1/013; A61L 15/08; A61L 15/12; A61L 15/14

(57) ABSTRACT

A folded compact inflatable structure has one or more chambers containing one or more folded metallic members, wherein the chambers and the metallic members are configured to unfold into an inflatable structure capable of being transformed by a foam material into a support or load bearing structure of a predefined shape and size. An orthopedic structure for supporting a limb in a subject has a folded non-inflated state and an unfolded inflated state in a form of an orthopedic brace. The structure includes one or more chambers provided with one or more metallic members. In the folded non-inflated state both the chambers and the metallic members are folded. The folded non-inflated state is transformable into the unfolded inflated state by unfolding and causing the metallic members to extend along a long axis of the chambers and cause an interior of the chambers to inflate with a foam material.

### RELATED APPLICATIONS

This Application is a Continuation-in-Part of PCT Application No. PCT/IL2024/050644, entitled “Foldable Inflatable Load Bearing Structure With Metal Support,” filed Jul. 3, 2024, which claims priority to Israeli Patent Application No. 304,279, filed Jul. 5, 2023, entitled “Foldable Inflatable Load Bearing Structure With Metal Support,” the contents of which are hereby incorporated by reference as if fully set forth herein.

### FIELD OF INVENTION

The invention is generally in the field inflatable structures and specifically foldable inflatable structures with a metal support.

### BACKGROUND

Portability, versatility, low weight, and immediate deployment are some of the reasons foldable and inflatable structures are used. Such advantages have prompted numerous manufacturers to explore alternatives in a form of inflatable products. The ability to quickly set-up or erect a structure provides a wide range of benefits to medical as well as to military personnel.

Typically, inflatable structures are structured of two sheets of a polymeric or a fabric material, such that the space between the sheets is pressurized with air or another gaseous material. Robust erected structures such as buildings and load-bearing structures require rigid non-inflatable spares or ribs. The presence of such spars or ribs, typically made of a metal, renders the inflatable structures less portable and heavier in weight.

Curable resin foams have been used in place of compresses air to achieve a robustness where metal spares or ribs could not be used or were undesired. Polyurethane has been used to fill a structure as stretcher poles or to immobilize patients by having a polyurethane foam filled structure surround body parts as it cures, but before it hardens to secure the body parts from movement.

In U.S. Pat. No. 4,744,116, a stretcher with hollow longitudinal tubes that are stretcher poles is provided separate from the litter bearing surface. The tubes are impregnated with a fluid curable resin and wrapped with a waterproof material. The tubes inflate when water is introduced and the resin hardens. The material cures and the tubes are used as stretcher poles.

In US Publication No. 2017/0258622, a position retaining device to stabilize patients is proposed. The device has a casing with containers having chemical precursors of a curable foam. The casing also has a plurality of beads. The containers are ruptured, the precursors mix, and the resulting polyurethane surrounds the beads molding the casing around the body part.

US Publication No. 2008173316 discloses a disposable spine support. The stretcher is flat and to provide spine support a bag within a bag is provided. The two bags have the precursors of a curable resin. When the inner bag is ruptured, the resulting reaction creates a curable foam that confirms to patient's spine.

US Publication No. 2021/368998 discloses a smart bed with transverse viscoelastic polyurethane foam members. The cylinders are surrounded by heaters to adjust firmness. The foam members have an adjustable stiffness to adjust hardness of the bed.

Despite the convenience of converting a foldable, portable, compact and light weight body into a functional structure by using a foam-forming material, such as a polyurethane, due to their brittleness, foam-based materials are less suitable for load-bearing applications. Presence of a metal frame, spars or ribs could improve the load-bearing capabilities; however, at the same time would make the product lose its foldability, compatibility and simplicity of transport.

### SUMMARY OF INVENTION

The inventor of the invention disclosed herein has developed a portable and compact inflatable platform capable of being transformed, within a matter of seconds, into a robust support unit or a load-bearing structure capable of carrying objects reaching hundreds of kilograms in weight. The inflatable platform, being shaped as a support unit, e.g., a medical support unit, or a structure for recreational, infrastructural or otherwise as a load-bearing unit, is shaped and sized to meet the intended use. The platform is generally formed of a material enclosure comprising an internal flexible metallic or polymeric member, which may be provided within an inflatable chamber or on its internal or external surface. When in the folded configuration, the structure is a compact and lightweight object that may be packed to be carried under human power. The folded structure may have a weight that is between several hundred grams to about 5 or so kilograms, depending on the size of the structure.

When the structure is unfolded, the flexible metal member snaps into a predetermined or predefined shape, position or orientation, which may be further subsequently manipulated into a desired shape, position or orientation. The inflatable chamber is then inflated or caused to be inflated by one or a plurality of materials to a predetermined volume or pressure. The plurality of materials may include one or more gaseous materials such as air or an inert gas, or reactive precursors that may be combined, upon unfolding of the structure or at any time thereafter, to generate a solid foam that fills and inflates the fabric chamber surrounding the internal flexible metal member. As the chamber and the structure as a whole inflates, the foam quickly hardens forming a rigid mass. The flexible metallic member increases the stiffens of the structure allowing it to bear more weight than that without the metallic member.

The invention thus provides a provision of a folded compact inflatable platform comprising a folded inflatable structure having a folded metallic (or polymeric) member, wherein the structure and the metallic member are configured to unfold into the inflatable structure that is capable of being transformed by inflation into a support or load bearing structure of a predefined shape and size.

In other words, a platform or a structure of the invention is provided in a folded form that is compact and is capable of being inflated. In this folded form, both the inflatable structure (having one or more inflatable chambers) and the metallic member contained within or associated with a surface of the structure are folded. Both the structure and the metallic member are predesigned to transform into an unfolded and ready-to-be-used structure. The transformation into the inflated structure is possible by unfolding the folded form in which both the structure and each of the inflatable chambers and the metallic member are substantially extended or spread or shaped around a body or an object such that inflating the chamber(s) with a gaseous material or a foam material fixes the structure in the desired shape and grants the structure the desired mechanical stiffness and robustness.

In some embodiments, the structure comprises one or more chambers or pockets that are formed between two material sheets or fabrics. For purposes of securing or maintaining mechanical integrity and further for isolating the content of each of the one or more chambers from leaking out, the one or more chambers are formed of one to several layers of a fabric or a material that endows the structure with sufficient mechanical strength and at times thermal insulation. The structure may be formed as a reusable pocket and each of the one or more chambers may be provided in a material that is disposable. The structure may be provided with a zipper or other closing means to hold the one or more chambers in place during inflation and use.

As used herein, the term “inflatable” is used to indicate a form of the structure that is capable of being filled with or inflated with a gaseous material or a foam material. The structure being filled with the gaseous or foam material is said to be inflated.

The invention provides a support or a load bearing structure having a folded non-inflated state and an unfolded inflated state, wherein the structure comprising one or more chambers embedded or provided with one or more metallic members, such that in the folded non-inflated state both the structure (including the one or more chambers) and the one or more metallic members are folded; the folded non-inflated state is transformable into the unfolded inflated state by unfolding and causing the one or more metallic members to extend a long an axis (typically, along the long axis) of any of the one or more chambers and by causing an interior of the one or more chambers to inflate with a gaseous or a foam material.

The invention further provides an inflatable support or load-bearing structure, the structure formed of an inflatable folded structure having one or more inflatable chambers embedding (comprising or associated with) one or more folded metallic members, said inflatable folded structure is transformable into the structure capable of supporting or load bearing by unfolding, causing the one or more metallic members to unfold and extend a long an axis of the one or more chambers and an interior of the one or more chambers to inflate or fill with a gaseous or a foam material (to a final predesigned structure or form).

In some embodiments, the unfolded structure is positioned around an object or a human limb or body part such that the metallic member(s) provide support to said object, limb or organ, thereafter the interior of the one or more chambers is inflated or filled with the gaseous or foam material.

The invention further provides a structure provided in a folded form, the structure comprising:
at least one folded inflatable chamber having an interior and an exterior; and
at least one folded flexible metallic member interior to said folded inflatable chamber;
wherein the interior of the inflatable chamber is inflatable to endow the structure with a required stiffness.

Further provided a folded structure, e.g., a load bearing structure, the structure comprising:
at least one folded inflatable chamber having an interior and an exterior;
at least one folded flexible metallic member interior to said foldable inflatable chamber; and
one or more curable resin precursor(s) configured, upon unfolding of the structure, to generate solid foam in the interior of the unfolded inflatable chamber.

The structure of the invention is designed as a single kit of a compact folded structure that can be easily carried and transformed into a functional structure upon demand, and without needing to resort to materials or operating tools that are not contained within the kit. Typically, any of the designs or structures of the invention (wherein each design is configured as a different supporting structure or a load bearing structure) is capable of being folded into a compact body that can be carried by human power or can be carried in a backpack or as a carry-on. The size of the compact folded structure may vary and will depend on the size of the structure, when in the inflated form, and the presence of any auxiliary unit that is mounted or associated therewith.

Any kit may comprise the folded structure, including the structure with one or more chambers and the metallic member(s), and optionally means for inflating the structure with a gaseous or a foam material. Where the structure is designed to be inflated with air, the kit may include a pump capable of inflating the structure. Where the structure is designed to be inflated with an inert gas, e.g., nitrogen, the kit may be provided with a vessel containing compressed inert gas. Where the structure is designed to be inflated with a foam material, the structure may be provided with a breakable capsule or two capsules or a plurality of capsules containing precursor materials that can combine to generate the foam.

As used herein, the term “folded” generally refers to a state wherein the structure and its parts are collapsed, bent, or rearranged or manipulated in form into a state that is smaller in size and into a shape that is compact and easily carried, and which can be reinstated when taking the opposite measure of unfolding. The term encompasses all forms of reducing in size the metallic member(s) or the structure as a whole, for the purpose of compaction. The term includes traditional folding as well as telescoping. The folding may involve bending the non-inflated structure inwards or in any other way. At times the folding involves rolling or bending inwards towards the longitudinal axis of the structure. Typically, the folded structure has the metallic member also folded in the same way, such that unfolding the structure causes unfolding of the metallic member. In some configurations, however, the structure and the internal metallic member are independently and differently folded. For instance, the structure may be folded by rolling, while the metallic member may be folded along a different axis. The structure may be folded by bending inwards, while the metallic member may be folded by rolling into a snail-shape.

In some embodiments, in the folded non-inflated state of the structure, the metallic member is provided in a rolled state (e.g., a snail-shape), in a form of a plurality of segments that can be extended and arranged to be locked into place, in a form of a plurality of hollow tubular sections slideably received within each other, in a form of a plurality of sections that slide out of a shaft and fold for compaction, in a form of a plurality if sections that can be assembled and secured by pins, rubber segments or any other way, and others.

The structure may be designed as a support structure or a load bearing structure that can carry large weights, e.g., a weight of a human being, over 100 Kg, without fracturing or breaking. Thus, the structure is predesigned, shaped and sized based on its intended use. The structure may be designed as a support structure, as a load-bearing structure or as any other structure. Generally, the structure may be used for land, ground, water, or any maritime application. It may be used as a floating device of any configuration and use.

The “support structure” may be any structure used to provide a mechanical support to an object. The support structure may be shaped as a pillar, a frame, a structural skeleton, etc, configured for use in a vertical or a horizontal position. In some embodiments, the structure may be used alone or in combination with like structures to form a modular structure. The modular structure may be used to provide a lightweight fluid impervious shelter or bivouac configured to be carried in a backpack.

The support structure may be an orthopedic member or brace that is configured and operable to support a broken body part or limb such as a leg, a hand, a neck, a finger, a toe etc.

The structure may be a “load-bearing structure” configured to support a weight of a subject or an object placed above. The structure may be shaped as a mattress or a stretcher or a flat support having an upper load bearing surface. According to some embodiments, the structure is provided with one or more or with a plurality of handles or straps that are attached to exterior sides of the structure to aid in carrying the inflated structure. The structure may also be provided with extendable legs or support members to position the structure above surface level; or wheels to easily maneuver the structure on a surface.

The structure may additionally be formed into a backboard to allow transportation of patients with suspected spinal injuries. It may have a flat hard top surface and may be provided with a plurality of handles to assist in transportation of patients.

In some embodiments, the structure can be used as a portable field surgical table.

In some embodiments, the structure may be used alone or in combination with like structures to form a modular structure. The modular structure may be used to provide a lightweight fluid impervious shelter or bivouac configured to be carried in a backpack.

The structure may alternatively be designed as a safety element such as a helmet, a protective or a support vest unit or a wearable clothing unit or a lifesaving vest unit.

In some embodiments, the structure may be a human size capsule for securing a human or an animal from environmental conditions.

The structure may alternatively be designed as an inflatable pad, tourniquet, a garment, a body protective unit, an umbrella, a parasol, a sleeping bag, a snowboard, a surfboard, a ladder, a cart, a tripod, a heating cover, a floating unit, a portable table or chair, and others, wherein each having at least one metallic member acting as a support element.

The invention further provides a structure having a folded non-inflated state and an unfolded inflated state, wherein the structure comprising one or more chambers embedded or provided with one or more metallic members, such that in the folded non-inflated state both the one or more chambers and the one or more metallic members are folded; the folded non-inflated state is transformable into the unfolded inflated state by unfolding and causing the one or more metallic members to extend a long an axis of the one or more chamber (or snap into position) and causing an interior of the one or more chamber to inflate with a gaseous or a foam material; the structure being any of the structures disclosed herein.

In some embodiments, the structure may be an orthopedic structure.

In some embodiments, the structure is a load bearing structure selected from a sleeping bag, a mattress, a ladder, a portable field surgical table, a stretcher, etc.

In some embodiments, the structure may be a human size capsule for securing a human or an animal from environmental conditions.

In some embodiments, the structure may be a wheel or a ring-shaped structure configured for mounting on a wheel axel. In such configurations, the metal member may be internal and/or external to said inflatable chamber.

The invention further provides an orthopedic device or an orthopedic brace having a folded non-inflated state and an unfolded inflated state, wherein the device or brace comprising one or more inflatable chambers embedded or provided with one or more metallic members, such that in the folded non-inflated state both the one or more chambers and the one or more metallic members are folded; the folded non-inflated state is transformable into the unfolded inflated state by unfolding and causing the one or more metallic members to extend a long an axis of the one or more chambers and by causing an interior of the one or more chambers to inflate with a gaseous or a foam material.

The structure comprises at least two sheets of a fabric material, the space between the two or more sheets defines an interior pocket or a “chamber” that is inflatable. The exterior faces of the structure may be sized and shaped based on the intended use. In some cases, the interior pocket or chamber may be smaller or of a different shape than the exterior shape of the structure. The structure may include one or more enclosed spaces or chambers, each capable of being individually inflated and each may independently be associated with or contain an individual metallic member.

In some embodiments, each structure comprises one or two inflatable chambers and one or two metallic members.

The structure may be formed of a flexible fluid- or gas-impervious material that is sutured or structured to define the size and shape of the chamber(s) volume. The fluid/gas impervious material may be one of a textile, plastic, polymeric, textile impregnated with waterproof material, laminated textile with waterproof material, or any other such material that is optionally light weight and sufficiently flexible or deformable to be folded into a compact structure.

Alternatively, the structure may comprise a disposable chamber capable of being inflated, wherein the disposable chamber may be formed of an impervious material and is discardable following use. The structure may be reused.

In some embodiments, the structure of the invention is provided with a single inflatable chamber, which size and shape may be defined by the exterior of the structure or by an internal wall structure. In other embodiments, the structure may comprise two or more chambers, each independently inflated and separated by walls defining their shapes and sizes. Depending on the number of chambers, their shapes and sizes, the shape, size and position of the metal member may be determined. For example, where two or more chambers are independently inflated, each may be provided or equipped with a separate metal member. Where a plurality of metal members is provided, they may be arranged to snap or unfold and aggregate into a form or structure of a predetermined shape. For example, a rod-like metal member may be provided in each of a plurality of chambers and are arranged to snap into position and structure to overall form a frame structure or an I-shape.

The one or more metallic members are flexible metallic members that can be folded or rolled within the folded structure such that upon unfolding, each of the metallic members snaps into or reassumes its shape. Where a single member is used, the member may be internal to a chamber or is associated with a wall of the chamber. In some cases, the metallic member is provided within a designated pocket that is within a chamber or formed on a wall region of the chamber.

Where two or more metallic members are present, at least one of the members is provided interior to a chamber. In some embodiments, a flexible metal member is also provided exterior to the chamber.

In the folded structure, the metal member is provided in a folded form that can spontaneously snap or convert into a predetermined shape or structure that provides an additional support to the structure, e.g., a load bearing structure. The predetermined shape of the metal member may vary based on the type of structure used, the load it is meant to carry, and other considerations known in the art.

In some cases, the predetermined shape may be a final shape of the metal member or may be further manipulated to adopt a final shape. Manipulation of the spontaneously converted member may be by a user by way of reshaping the member or repositioning the member within the chamber. Manipulation may also comprise securing or associating the member in its predetermined shape to any part of the structure.

The one or more metallic members may convert into a frame structure that occupies the internal circumference of the chamber, into a rib structure, into an I shape, into a T shape, or into any other shape. The metallic member(s) may also convert into a cylindrical or a tube shape having a predefined inner diameter and cross-sectional shape (oval, circular, spheroidal, etc). To allow for a spontaneous conversion from a folded shape into an unfolded or extended shape, the flexible metallic member may be manufactured from a shape memory metal or alloy, or from a metal member which was shaped and configured to change form and shape upon demand or upon application of a stimulus such as a mechanical force, a temperature, exposure to water, etc. The use of a metal or an alloy such as a shape memory metal or alloy allows the flexible metallic member to snap, that is to rapidly assume the predetermined shape without human assembly.

In some embodiments, the flexible metallic member or members are oriented within the chamber at an oblique angle to the longitudinal axis of the chamber.

In some embodiments, a plurality of metallic members is provided, wherein the members are aligned skew to each other not intersecting and not parallel; and wherein optionally the flexible metallic members are not in the same plane.

In some embodiments, the metallic member is formed of a metal or a metal alloy of any metal having sufficient flexibility and mechanical memory. Such metals and metal alloys include aluminum; alloys of copper; zinc and aluminum alloys (Cu—Zn—Al alloys); copper, aluminum and nickel alloys (Cu—Al—Ni alloys); iron, manganese, silicon alloys (Fe—Mn—Si alloys); stainless steel, brass metal, and others.

In some embodiments, the metallic member is an aluminum member.

In some embodiments, the metallic member is stainless steel.

The thickness and other dimensions of the metallic member may vary.

For example, the member may be of a thickness between 0.2 mm and 2 cm. The length and width of the member may change based on the structure to be reinforced. In some cases, the metallic member may snap into a tubular form having a diameter depending on the width of the member.

In some embodiments, the metallic member is of a metal or a metal alloy, as disclosed herein, that may be rolled into a snail-shape and can snap into an elongated shape or tubular shape upon unfolding of the structure. The tubular shape endows the structure with sufficient rigidity that can sustain a human weight.

In some configurations, the metal member may be replaced by or used in combination with a member of a different material such as reinforcing fiberglass, plastics and polymers and others.

In some cases, the structure of the invention is provided with a precursor of a curable resin that can easily and expeditiously form into a foam material upon demand. Typically, the precursor may comprise a material or two or more materials that can convert into or interact to form a foam material that occupies the full volume of the chamber in which the precursor was provided. To prevent spontaneous reactivity, the precursor(s) is/are kept in an enclosed pod or container that must be manually induced to generate the foam. In cases where the precursor is a pair or a plurality of materials which in combination react to form the foam, the two or more materials may be separately contained within a common pod or sack, which can be ruptured or caused to break, to thereby bring about contact between the precursors and evolution of a foam material. In other words, when the pod or sack comprising the two or more precursors is ruptured, the curable resin precursors are combined, and the curable resin forms a foam that inflates the foldable inflatable chamber.

In some embodiments, the precursors are provided in a cartridge and are combined when the cartridge valve is opened forming a foam. In such configurations, the cartridge may be in fluid communication with the inflatable chamber(s) through an inlet so that the curable resin foam is injected into the foldable chamber, inflating the chamber. The curable resin hardens into a mass and even before it is fully cured, it is cable of supporting a load on the chamber's upper surface.

In some embodiments, the curable resin is a polyurethane. In other words, the foam formed in the structure is polyurethane or a homolog or derivative thereof. Similarly, the resin may be of any foam-forming material.

Where the foam is formed of polyurethane, the precursor used may be at least one isocyanate prepolymer and at least one polyol. The two components may be separably contained and may each, or one of which, be provided with a catalyst in a form of a blowing agent, such as an amine. The isocyanate and polyol may be selected as known in the art of foam manufacturing. Exemplary isocyanates include methylene diphenyl diisocyanate (MDI), toluene diisocyanate (TDI), hexamethylene diisocyanate (HDI) and isophorone diisocyanate (IPDI). Exemplary polyols may include polyether polyols such as polyethylene glycol, polytetramethylene ether glycol, and others.

The invention further provides a kit or a commercial package comprising a foldable structure according to the invention and at least one life-support unit such as a defibrillator, a breathing unit, an oxygen generating or providing unit, a ventilator and others. The kit may also comprise instructions of use.

In some embodiments, the foldable structure may be any load bearing unit, an orthopedic brace, mattress, stretcher, protective capsule, and others as disclosed herein.

The invention further contemplates a method of operating an inflatable structure having a folded non-inflated state and an unfolded inflated state, and comprising one or more inflatable chambers embedded or provided with one or more metallic members, the method comprising unfolding the structure in the folded non-inflated state causing the one or more metallic members to snap and extend a long an axis of the unfolded one or more chambers; and causing an interior of the one or more chambers to inflate with a gaseous or a foam material to transform into a support structure or a load bearing structure.

In some embodiments, the structure is a structure according to the invention.

The unfolding of the folded non-inflated structure may be achieved manually by a user operating or using the structure. The unfolding may be achievable by removing the folded structure from a carrier or by permitting the folded structure to snap into an unfolded position by the unfolding or snapping of the metallic member(s) into position.

The unfolding typically causes or involves snapping into position of the metallic member(s). In some embodiments, the metallic member spontaneously snaps into a predetermined shape or structure.

In some embodiments, the structure may be an orthopedic brace and the unfolding may comprise positioning the unfolded non-inflated structure around a body part and allowing the structure to fill with a gaseous or a foam material to take shape around the body part.

In some embodiments, upon unfolding, the metallic member spontaneously snaps. In other configurations, upon unfolding, the metallic member is manually transformed into an elongated metallic member that takes a position within the chamber.

In some embodiments, one or more of the metallic members is arranged such that snapping from the folded to an unfolded state is along a long axis of the structure.

In some embodiments, upon unfolding the structure spontaneously fills with a gaseous or a foam material, wherein, for example, the unfolding causes a pump unit to inflate the chambers with air; or wherein, for example, the unfolding causes a foam precursor material(s) to generate a foam material in the chambers.

Alternatively, upon folding air is caused to fill the one or more chambers; or a capsule comprising a foam precursor(s), as explained herein, is mechanically caused to break (e.g., by a user) to generate the foam material.

The invention further provides:

A folded compact inflatable structure comprising a structure having one or more chambers containing one or more folded metallic members, wherein the one or more chambers and the one or more metallic members are configured to unfold into an inflatable structure capable of being transformed by a foam material into a support or load bearing structure of a predefined shape and size.

In some configurations of structures of the invention, the structure having one or more chambers and a metallic member contained in one of the chambers, wherein the one or more chambers are configured upon unfolding of the metallic member to fill with a foam material.

In some configurations of structures of the invention, being a support or a load bearing structure having a folded non-inflated state and an unfolded inflated state, wherein the structure comprising one or more chambers provided with one or more metallic members, such that in the folded non-inflated state both the one or more chambers and the one or more metallic members are folded; the folded non-inflated state is transformable into the unfolded inflated state by unfolding and causing the one or more metallic members to extend a long an axis of the chambers and causing an interior of the one or more chamber to inflate with a foam material.

In some configurations of structures of the invention, provided in a kit of elements, wherein the kit comprises the folded structure, including the one or more chambers and the metallic member(s), and means for inflating the structure with the foam material.

In some configurations of structures of the invention, in the unfolded inflated state the structure is a support structure designed to provide a mechanical support to an object.

In some configurations of structures of the invention, in the unfolded inflated state the support structure is shaped as a pillar, a frame, a structural skeleton, or a modular unit.

In some configurations of structures of the invention, in the unfolded inflated state the support structure is an orthopedic member or brace.

In some configurations of structures of the invention, in the unfolded inflated state the support structure is an orthopedic member or brace configured and operable to support a broken body part.

In some configurations of structures of the invention, in the unfolded inflated state the structure is a load-bearing structure configured to support a weight of a subject or an object placed above.

In some configurations of structures of the invention, in the unfolded inflated state the load-bearing structure is provided with one or more or with a plurality of handles or straps that are attached to exterior sides of the structure to aid in carrying the inflated structure.

In some configurations of structures of the invention, in the unfolded inflated state the load-bearing structure is provided with extendable legs or support members to position the structure above surface level; or wheels to maneuver the structure on a surface.

In some configurations of structures of the invention, in the unfolded inflated state the load-bearing structure is a backboard configured to transport of a human or an animal subject.

In some configurations of structures of the invention, in the unfolded inflated state the load-bearing structure is a mattress or a stretcher or a table, or a portable field surgical table.

In some configurations of structures of the invention, in the unfolded inflated state the structure is a helmet, or a protective or a support vest unit or a wearable clothing unit or a lifesaving vest unit.

In some configurations of structures of the invention, in the unfolded inflated state the structure is a capsule for securing a human or an animal from environmental conditions.

In some configurations of structures of the invention, in the unfolded inflated state the structure is an inflatable pad, tourniquet, a garment, a body protective unit, an umbrella, a parasol, a sleeping bag, a snowboard, a surfboard, a ladder, a cart, a tripod, a heating cover, a floating unit, a portable table or chair.

In some configurations of structures of the invention, in the unfolded inflated state the structure is a load bearing structure selected from a sleeping bag, a mattress, a ladder, a portable field surgical table, or a stretcher.

A load-bearing structure for carrying a human, the structure having a folded non-inflated state and an unfolded inflated state in a form of a stretcher, wherein the structure comprising one or more chambers provided with one or more metallic members, such that in the folded non-inflated state both the one or more chambers and the one or more metallic members are folded; the folded non-inflated state is transformable into the unfolded inflated state by unfolding and causing the one or more metallic members to extend a long a long axis of the chambers and causing an interior of the one or more chambers to inflate with a foam material.

An orthopedic structure for supporting a limb in a subject, the structure having a folded non-inflated state and an unfolded inflated state in a form of an orthopedic brace, wherein the structure comprising one or more chambers provided with one or more metallic members, such that in the folded non-inflated state both the one or more chambers and the one or more metallic members are folded; the folded non-inflated state is transformable into the unfolded inflated state by unfolding and causing the one or more metallic members to extend a long a long axis of the chambers and causing an interior of the one or more chambers to inflate with a foam material.

In some configurations of structures of the invention, the structure is formed of at least two sheets of a fabric material, the space between the at least two sheets forming the one or more chambers.

In some configurations of structures of the invention, the structure is formed of at least two sheets of a fabric material, forming a thermally insulating structure.

In some configurations of structures of the invention, the structure is configured to receive and hold one or more disposable chambers of an impervious material, said chambers being configured to comprising the foam material.

In some configurations of structures of the invention, the structure comprising a single chamber or two or more chambers, each independently inflated and separated by walls defining their shapes and sizes.

In some configurations of structures of the invention, the one or more metallic members is/are a flexible metallic member capable of being folded or rolled within the folded structure such that upon unfolding the one or more metallic members snap into or reassume their shape.

In some configurations of structures of the invention, in the folded structure, the one or more metallic member is provided in a folded form capable of spontaneously snaping or converting into a predetermined shape or structure.

In some configurations of structures of the invention, the one or more metallic members is/are capable of spontaneously snaping or converting into a frame structure that occupies the internal circumference of the chamber, into a rib structure, or into an I shape.

In some configurations of structures of the invention, the one or more metallic members is/are formed of a metal or a metal alloy selected from aluminum; alloys of copper; zinc and aluminum alloys (Cu—Zn—Al alloys); copper, aluminum and nickel alloys (Cu—Al—Ni alloys); iron, manganese, silicon alloys (Fe—Mn—Si alloys); stainless steel, or brass.

In some configurations of structures of the invention, the one or more metallic members is an aluminum member or a stainless-steel member.

In some configurations of structures of the invention, the structure is provided with a precursor of a curable resin configured to form a foam material upon demand.

In some configurations of structures of the invention, the precursor is provided in a pod or container.

In some configurations of structures of the invention, the precursor is a pair or a plurality of different materials which in combination react to form the foam.

In some configurations of structures of the invention, the different materials are separately contained within a common pod configured to be ruptured or caused to break, to permit contact between the different materials and form the foam material.

In some configurations of structures of the invention, the pod is ruptured manually.

In some configurations of structures of the invention, the foam material is a curable resin.

In some configurations of structures of the invention, the curable resin is a polyurethane.

In some configurations of structures of the invention, the different materials comprise at least one isocyanate prepolymer and at least one polyol.

In some configurations of structures of the invention, the polyurethane is formed of at least one isocyanate prepolymer and at least one polyol.

A method of operating an inflatable structure having a folded non-inflated state and an unfolded inflated state, and comprising one or more chambers provided with one or more metallic members, the method comprising unfolding the structure in the folded non-inflated state causing the one or more metallic members to snap and extend a long an axis of the unfolded chambers; and causing an interior of the one or more chambers to inflate with a foam material to transform into a support structure or a load bearing structure.

In some configurations of methods of the invention, the structure is an orthopedic brace and the unfolding may comprise positioning the unfolded non-inflated structure around a body part and allowing the structure to fill with the foam material to take shape around the body part.

In some configurations of methods of the invention, the one or more metallic members is/are arranged such that snapping from the folded to an unfolded state is along a long axis of the structure.

In some configurations of methods of the invention, upon unfolding, the structure spontaneously fills with the foam material.

A kit or commercial package comprising a folded structure having a folded non-inflated state and an unfolded inflated state, wherein the structure comprising one or more chambers provided with one or more metallic members, such that in the folded non-inflated state both the one or more chambers and the one or more metallic members are folded; the folded non-inflated state is transformable into the unfolded inflated state by unfolding and causing the one or more metallic members to extend a long an axis of the chambers and by causing a foam precursor material to generate a foam material, thereby inflating the chamber.

### BRIEF DESCRIPTION OF THE DRAWINGS

FIGS. 1A-B are images of folded non-inflated structure according to some embodiments of the invention. The folded structure is provided in a size fitting a regular backpack.

FIG. 2 is a view of an internal wall section of a chamber showing position of a metallic element according to some embodiments of the invention.

FIGS. 3A-D are different views of an inflated stretcher according to some embodiments of the invention.

FIG. 4 is a depiction of an inflated stretcher provided with side handles for carrying a human subject according to some embodiments of the invention.

FIGS. 5A-C show an orthopedic support unit folded under a leg of a human (A), folding of the support unit over the leg of the human (B), and inflating the unit over the leg, as disclosed herein.

### DETAILED DESCRIPTION OF THE INVENTION

The technological problem is to provide a foldable portable structure that can provide an added support or robustness or which can bear a heavy load, while being carried out to a field with relative ease. The solution is to provide a structure as disclosed herein, having a metallic member(s) which is provided in a folded configuration and which is yet configured to snap into a predetermine shape.

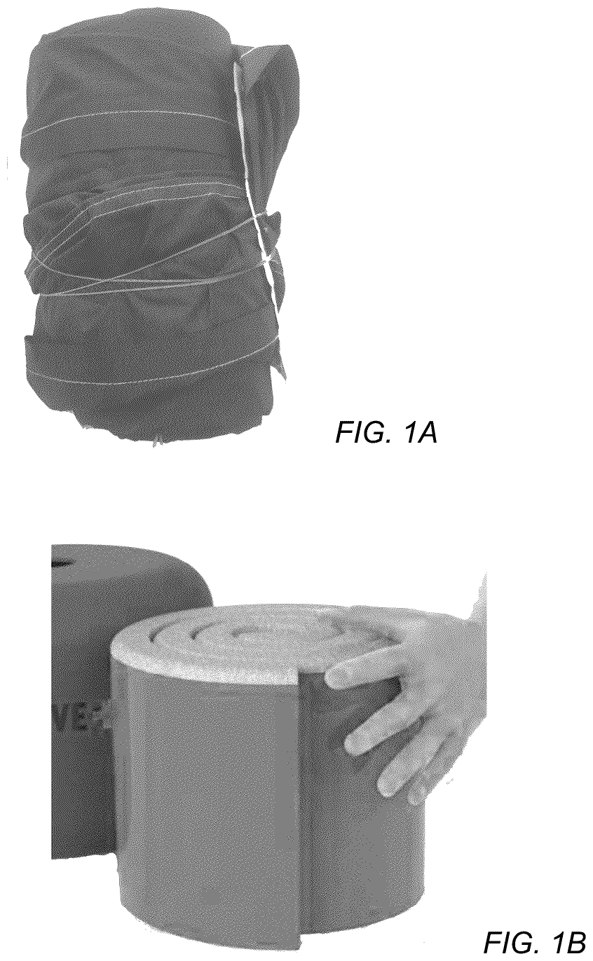

FIG. 1A shows the structure in a folded and secured form. In the folded configuration it can be carried in a backpack under human power to the field. In the configuration shown in FIG. 1B, the structure is folded by rolling the fabric structure including the metallic member into a cylinder shape.

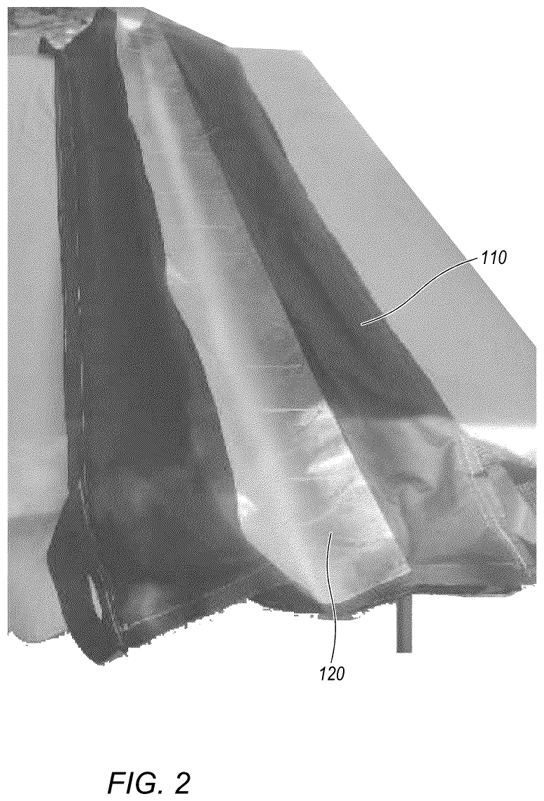

FIG. 2 is a view of an unfolded structure, showing an internal wall of the chamber. The internal wall 110 is unfolded showing the metallic member 120 has taken its predetermined shape upon unfolding. The flexible metal member 120 is formed from stainless steel.

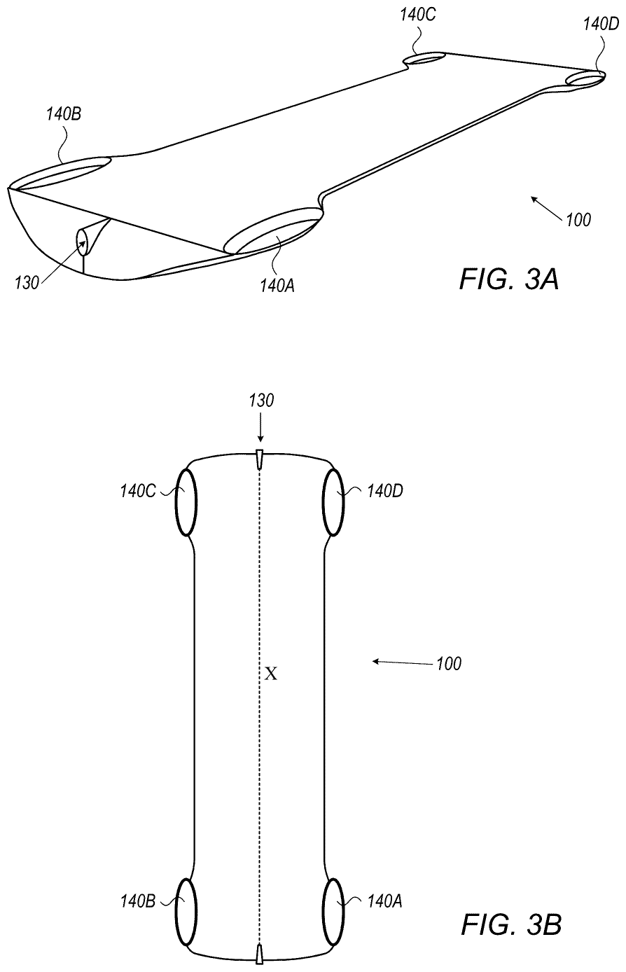

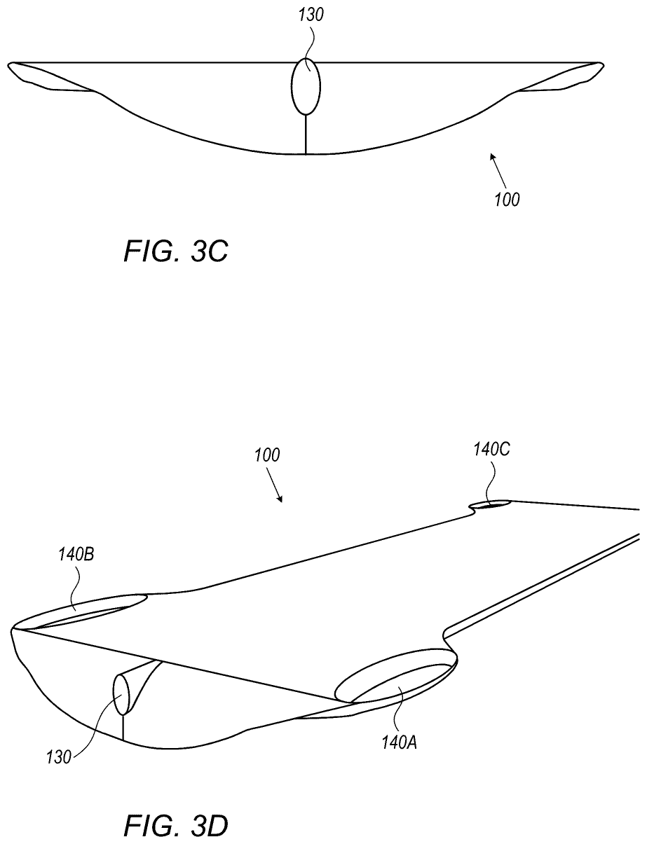

FIGS. 3A-D show different views of an unfolded structure 100 designed as a stretcher, wherein the chamber is filled with a polyurethane form endowing the stretcher with the needed robustness and rigidity. The metallic member 130 extending along the stretcher's long axis X is provided interior to the chamber. As shown, when snapping into position, upon unfolding, the metallic member 130 has assumed the predetermined shape of a tube. The predetermined shape may comprise a cross-sectional shape of the tube that is at least one of a circle, an oval, and an ovoid. One advantage of a predetermined shape is that it may be selected for a particular use. The various shapes can be selected for strength or the use of the structure. If the structure is to be used as a stretcher, as depicted, the tube shape may be advantageous in supporting a patient as the load will be vertical to the ground. If the use is as a structure for shelter, the shape can be selected to minimize deflection of the structure when used alone or in combination with other like structures.

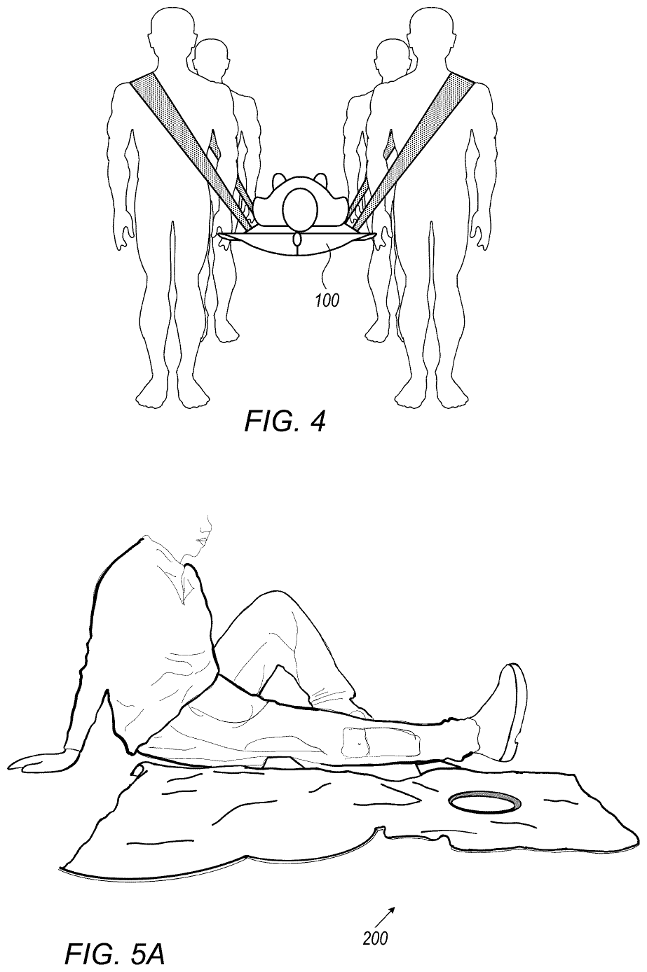

On the sides of the structure 100 are handles 140A, 140B, 140C and 140D. The number of handles may vary and be attached or formed on the perimeter of structure 100. The handles can be attached or are integral to the structure 100. The handles can be configured to receive straps for carrying the structure (as exemplified in FIG. 4).

FIG. 4 is a depiction of a stretcher structure 100 carried by 4 people.

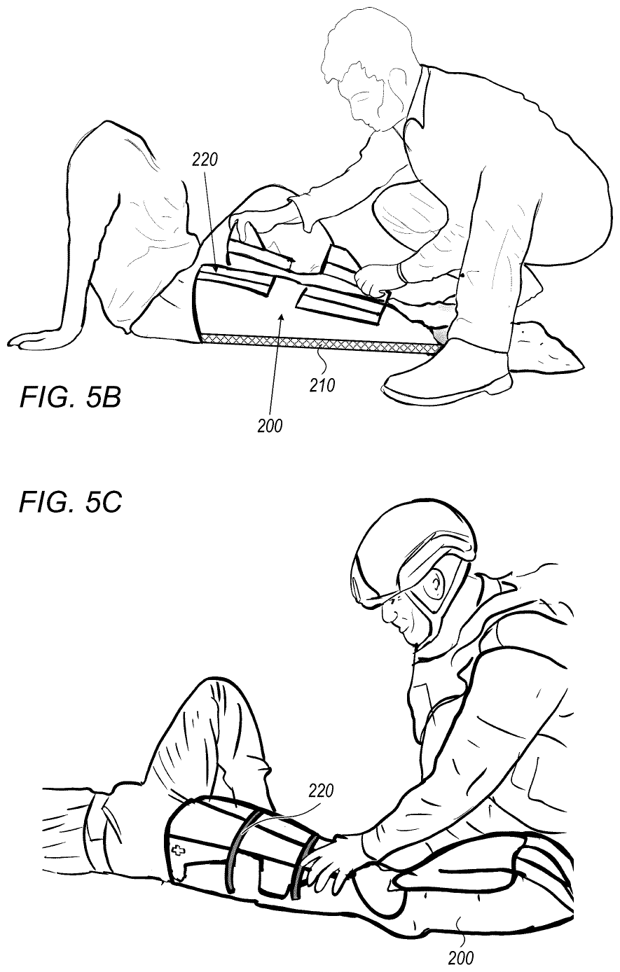

FIGS. 5A-C are depictions of a human fitted with a leg brace for securing and stabilizing a broken limb. FIG. 5A shows the orthopedic structure 200 in an unfolded form, wherein the metallic member (not shown) extends the length of the structure. The structure 200 is folded around the leg before foam precursor materials are caused to form a foam within a single chamber of the structure. The dressing of the leg is assisted by securing means, such as fasteners 220 that ensure tight fit around the leg. The structure is positioned below the subject's leg such that the metallic member 210 being internal to the chamber is positioned below the leg, along the long axis of the structure, as shown in FIG. 5B. Once the structure holds the leg in place, a capsule of the foam precursors provided in the structure chamber is broken, permitting formation of a foam within the structure (FIG. 5C).

The foam may be formed through a chemical reaction of two precursor materials. For example, the foam may be a curable resin, such as a polyurethane. In such cases, the two precursor materials may be an isocyanate prepolymer and a polyol. Alternatively, the precursor materials may be any other suitable precursor materials that may combine to form a curable resin. The two precursor materials may be stored in separate capsules which may open into a common chamber. After breaking of the capsules, the user may mix them manually within the common chamber, to generate the curing reaction.

The curing of the foam within the structure may be a highly exothermic reaction. The heat that is released from the area in which the foam is formed may be injurious to the patient's limb, and at minimum may be uncomfortable. Accordingly, it is desirable, in embodiments in which foam curing occurs, to insulate the part of the body adjacent to the leg brace from the area of foam formation. This insulation may be achieved through use of multiple layers of fabric material which may form a chamber therebetween and which intervene between the location of foam curing and the leg. The fabric in between the location of the reaction and the limb may be made thicker, or otherwise more insulating, than the fabric on the exterior face of the structure. In addition, the orthopedic structure may be equipped with ventilation holes, for example, on the exterior face of the chamber in which the chemical reaction occurs, to assist in release of heat from the location of curing to the ambient atmosphere.

It will be appreciated that the embodiments described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. An orthopedic structure for supporting a limb in a subject, the structure having a folded non-inflated state and an unfolded inflated state in a form of an orthopedic brace, wherein the structure comprising one or more chambers provided with one or more metallic members, such that in the folded non-inflated state both the one or more chambers and the one or more metallic members are folded; the folded non-inflated state is transformable into the unfolded inflated state by unfolding and causing the one or more metallic members to extend along a long axis of the chambers and causing an interior of the one or more chambers to inflate with a foam material.

2. The structure according to claim 1, formed of at least two sheets of a fabric material, the space between the at least two sheets forming the one or more chambers.

3. The structure according to claim 1, further comprising a thermally insulating structure arranged between the one or more chambers and an interior portion of the structure that is configured to be adjacent to the limb.

4. The structure according to claim 1, further comprising ventilation holes, the ventilation holes configured to convey heat from a curing of the foam material to ambient atmosphere.

5. The structure according to claim 1, wherein the structure is configured to receive and hold one or more disposable chambers of an impervious material, said chambers being configured to comprise the foam material.

6. The structure according to claim 1, wherein the one or more metallic members is/are formed of a metal or a metal alloy selected from aluminum; alloys of copper; zinc and aluminum alloys (Cu—Zn—Al alloys); copper, aluminum and nickel alloys (Cu—Al—Ni alloys); iron, manganese, silicon alloys (Fe—Mn—Si alloys); stainless steel, or brass.

7. The structure according to claim 1, provided with a precursor of a curable resin configured to form the foam material upon demand.

8. The structure according to claim 7, wherein the precursor is formed in a pod or container.

9. The structure according to claim 7, wherein the precursor is a pair or plurality of different materials which in combination react to form the foam, and wherein the different materials are separately contained within a common pod configured to be ruptured or cause to break, to permit contact between the different materials and form the foam material.

10. The structure according to claim 9, wherein the different materials comprise at least one isocyanate prepolymer and at least one polyol.

11. A folded compact inflatable structure comprising a structure having one or more chambers containing one or more folded metallic members, wherein the one or more chambers and the one or more metallic members are configured to unfold into an inflatable structure capable of being transformed by a foam material into a support or load bearing structure of a predefined shape and size.

12. The structure according to claim 11, provided in a kit of elements, wherein the kit comprises the folded structure, including the one or more chambers and the one or more metallic members, and means for inflating the structure with the foam material.

13. The structure of claim 11, wherein in the unfolded inflated state the structure is a support structure designed to provide a mechanical support to an object.

14. The structure of claim 13, wherein in the unfolded inflated state the support structure is shaped as a pillar, a frame, a structural skeleton, or a modular unit.

15. The structure of claim 13, wherein in the unfolded inflated state the support structure is an orthopedic member or brace.

16. The structure according to claim 11, being a support or a load bearing structure having a folded non-inflated state and an unfolded inflated state, wherein the structure comprises the one or more chambers provided with the one or more metallic members, such that in the folded non-inflated state both the one or more chambers and the one or more metallic members are folded; and wherein the folded non-inflated state is transformable into the unfolded inflated state by unfolding and causing the one or more metallic members to extend along an axis of the chambers and causing an interior of the one or more chambers to inflate with the foam material.

17. The structure of claim 16, wherein in the unfolded inflated state the load-bearing structure is provided with one or more handles or straps that are attached to exterior sides of the structure to aid in carrying the inflated structure, and/or extendable legs or support members to position the structure above surface level; and/or wheels to maneuver the structure on a surface.

18. The structure of claim 11, wherein in the unfolded inflated state the structure is a backboard, mattress, stretcher, table, portable field surgical table, helmet, protective or support vest unit, wearable clothing unit, lifesaving vest unit, a capsule for securing a human or an animal from environmental conditions, an inflatable pad, tourniquet, garment, body protective unit, umbrella, parasol, sleeping bag, snowboard, surfboard, ladder, cart, tripod, heating cover, floating unit, portable table or chair, sleeping bag, mattress, or ladder.

19. A method of operating an inflatable structure having a folded non-inflated state and an unfolded inflated state, and comprising one or more chambers provided with one or more metallic members, the method comprising unfolding the structure in the folded non-inflated state causing the one or more metallic members to snap and extend along an axis of the unfolded chambers; and causing an interior of the one or more chambers to inflate with a foam material to transform into a support structure or a load bearing structure.

20. The method according to claim 19, wherein the structure is an orthopedic brace and the unfolding comprises positioning the unfolded non-inflated structure around a body part and allowing the structure to fill with the foam material to take shape around the body part.
